(19) United States
(12) Patent Application Publication
(10) Pub. No.: US 2022/0331558 A1
(43) Pub. Date: Oct. 20, 2022

(54) DEVICES FOR USE IN INTERVENTIONAL AND SURGICAL PROCEDURES AND METHODS OF USE THEREOF

(71) Applicant: BODY VISION MEDICAL LTD., Ramat Hasharon (IL)

(72) Inventors: Dorian AVERBUCH, Ramat Hasharon (IL); Eliron AMIR, Ramat Hasharon (IL); Boaz HARARI, Ramat Hasharon (IL); Dima SEZGANOV, Ramat Hasharon (IL); Ronen NEEMAN, Ramat Hasharon (IL)

(21) Appl. No.: 17/763,747

(22) PCT Filed: Aug. 14, 2020
(86) PCT No.: PCT/IB20/00676

§ 371 Date: Mar. 25, 2022

Related U.S. Application Data
(60) Provisional application No. 62/888,288, filed on Aug. 16, 2019.

(51) Int. Cl. A61M 25/01; A61B 90/00

(52) U.S. Cl. CPC A61M 25/0138; A61M 25/0147; A61B 90/37; A61B 90/39

(57) ABSTRACT

A device including a sheath having a proximal end, a distal end, and a lumen extending through the sheath from the proximal end to the distal end, wherein the sheath is biased to a released position; a pull wire extending along the sheath and being coupled to the sheath at the distal end, wherein the pull wire and the sheath are configured to cooperate such that pulling the pull wire toward the proximal end causes the distal end to assume an active position, and such that release of the pull wire causes the distal end to return to the released position; and a control portion coupled to the proximal end of the sheath and to the pull wire, wherein the control portion includes a control element operable to selectively pull the pull wire toward the proximal end of the sheath or to release the pull wire.

### CROSS-REFERENCE TO RELATED APPLICATION

This application is an international (PCT) patent application relating to and claiming the benefit of commonly-owned, co-pending U.S. Provisional Patent Application No. 62/888,288, filed on Aug. 16, 2019, entitled “DEVICES FOR USE IN INTERVENTIONAL AND SURGICAL PROCEDURES AND METHODS OF USE THEREOF,” the contents of which are incorporated herein by reference in their entirety.

### FIELD

The present invention relates to medical imaging. More particularly, the present invention relates to a device that is configured to attach to a distal end of a bronchoscope, to enable navigation of the device when the device is positioned within a patient's body, and to enable determination of the depth of the device based on a two-dimensional medical image showing the device positioned within the patient's body. The present invention also relates to a method for using such a device.

### BACKGROUND

Bronchoscopes are medical devices that are used to obtain images of body cavities within the body of a patient (e.g., within a patient's lung). To properly evaluate the images obtained using a bronchoscope, the position of the bronchoscope in three dimensions (i.e., including the depth of the bronchoscope within the body) must be known.

### SUMMARY

In an embodiment, a device configured to be attached to a bronchoscope includes an applicator, a shaft, a catheter, a guide wire, a connector, a handle, and a radio opaque material, the applicator having a proximal end, a distal end, and an internal channel extending from the proximal end to the distal end, the shaft having a proximal end, a distal end, and an internal channel extending from the proximal end to the distal end, the shaft being configured to be slidably received within the internal channel of the applicator, the catheter configured to be positioned within the internal channel of the shaft, the guide wire positioned within the catheter, the connector configured to be attached to the distal end of the applicator, configured to engage a bronchoscope, and configured so as to be rotatable with respect to the shaft, the handle attached to the proximal end of the applicator, the handle comprising a trigger operable to selectively lock or unlock sliding motion of the shaft with respect to the applicator, the radio opaque material attached to an outer portion of the device, the radio opaque material being positioned in a predetermined pattern.

In an embodiment, the pattern is non-uniform. In an embodiment, the pattern includes the radio opaque material having a first density at a first location and a second density at a second location, the first and second densities being different from one another. In an embodiment, the radio opaque material is positioned (a) on the catheter, (b) on the guide wire, or (c) on both the catheter and the guide wire.

In an embodiment, the proximal end of the applicator includes a luer lock entrance. In an embodiment, the connector includes a luer lock plug that is connected to the luer lock entrance of the proximal end of the applicator.

In an embodiment, the guide wire is either flexible, rigid, pre-curved, and or configured to be curved. In an embodiment, the catheter includes a pull wire that is configured to control a curvature of the guide wire. In an embodiment, the grip handle is configured to rotate with respect to the shaft. In an embodiment, the device also includes a polytetrafluoroethylene tube positioned within the shaft and configured to guide movement of the catheter.

In an embodiment, a method for medical imaging includes providing a bronchoscope; the method also including providing a device configured to be attached to the bronchoscope, the device including an applicator, a shaft, a catheter, a guide wire, a connector, a handle, and a radio opaque material, the applicator having a proximal end, a distal end, and an internal channel extending from the proximal end to the distal end, the shaft having a proximal end, a distal end, and an internal channel extending from the proximal end to the distal end, the shaft being configured to be slidably received within the internal channel of the applicator, the catheter configured to be positioned within the internal channel of the shaft, the guide wire positioned within the catheter, the connector configured to be attached to the distal end of the applicator, configured to engage a bronchoscope, and configured so as to be rotatable with respect to the shaft, the handle attached to the proximal end of the applicator, the handle comprising a trigger operable to selectively lock or unlock sliding motion of the shaft with respect to the applicator, the radio opaque material attached to an outer portion of the device, the radio opaque material being positioned in a predetermined pattern; the method also including attaching the device to the bronchoscope; the method also including placing the bronchoscope within a body cavity of a body of a patient; the method also including obtaining at least one medical image of at least a portion of the body of the patient, the at least a portion including the body cavity; and the method also including determining a depth of the device within the body based on at least the predetermined pattern and the at least one medical image.

In an embodiment, the medical image is an X-ray.

In some embodiments, a device includes a sheath having a proximal end, a distal end opposite the proximal end, and a lumen extending through the sheath from the proximal end to the distal end, wherein the sheath is biased to a released position; a pull wire extending along the sheath from the proximal end to the distal end and being coupled to the sheath at the distal end, wherein the pull wire and the sheath are configured to cooperate such that pulling the pull wire toward the proximal end of the sheath causes the distal end of the sheath to assume an active position, and such that release of the pull wire causes the distal end of the sheath to return to the released position; and a control portion coupled to the proximal end of the sheath and to the pull wire, wherein the control portion includes a control element operable to selectively pull the pull wire toward the proximal end of the sheath or to release the pull wire.

In some embodiments, the sheath includes a plurality of radiopaque markers. In some embodiments, the plurality of radiopaque markers are arranged in a pattern along the sheath.

In some embodiments, the sheath is sized and shaped to be received within a bronchoscope having a working channel with a diameter of 2.8 mm and to be able to receive within the sheath of the lumen an endo-therapy accessory that is configured to fit within a 2.0 mm inside diameter working channel.

In some embodiments, the released position is a straight position and the active position is a curved position. In some embodiments, a curvature of the curved position is variable depending on an extent to which the pull wire is pulled toward the proximal end of the sheath.

In some embodiments, the control portion includes a lever operable by a user to pull the pull wire toward the proximal end of the sheath. In some embodiments, the device also includes a locking mechanism operable by a user to lock the lever in a selected position.

In some embodiments, the control portion also includes a luer lock configured to receive a syringe and to couple the syringe to the sheath.

In some embodiments, the device also includes a handle connection mechanism configured to couple the device to an applicator.

In some embodiments, a method includes (1) providing a device including a sheath, a pull wire, and a control portion, wherein the sheath includes a proximal end, a distal end opposite the proximal end, a lumen extending through the sheath from the proximal end to the distal end, wherein the sheath is biased to a released position, and wherein the sheath includes a plurality of radiopaque markers positioned along the sheath; wherein the pull wire extends along the sheath from the proximal end to the distal end and is coupled to the sheath at the distal end, wherein the pull wire and the sheath are configured to cooperate such that pulling the pull wire toward the proximal end of the sheath causes the distal end of the sheath to assume an active position, and such that release of the pull wire causes the distal end of the sheath to return to the released position; and wherein the control portion is coupled to the proximal end of the sheath and to the pull wire, wherein the control portion includes a control element operable to selectively pull the pull wire toward the proximal end of the sheath or to release the pull wire; (2) advancing the sheath into a body cavity of a patient so that the distal end of the sheath is positioned at a bifurcation within the body cavity; (3) displaying a view of the sheath within the body cavity by a real-time medical imaging modality obtained with a medical imaging device; (4) determining an optimal position of the distal end of the sheath to advance the sheath past the bifurcation; (5) operating the control portion to position the distal end of the sheath at the optimal position; and (6) advancing the sheath past the bifurcation.

In some embodiments, the method also includes the steps of determining an optimal pose of the medical imaging device to display the sheath and the bifurcation; positioning the medical imaging device at the optimal pose; and displaying an updated view of the sheath and the bifurcation, wherein the optimal position is determined based on the updated view.

In some embodiments, the body cavity is a bronchial airway.

In some embodiments, the method also includes repeating steps (3), (4), (5), and (6) at a further bifurcation. In some embodiments, steps (3), (4), (5), and (6) are repeated at further bifurcations until the distal end of the sheath reaches a target area.

### BRIEF DESCRIPTION OF THE DRAWINGS

FIG. 1 shows a flowchart of an exemplary method.

FIG. 2A shows a plot of density of radio opaque material along the length of an exemplary device.

FIG. 2B shows a plot of grayscale intensity in a fluoroscopic image of the device of FIG. 2A.

FIG. 2C shows a plot of grayscale intensity in a fluoroscopic image of the device of FIG. 2A with the device partially occluded.

FIG. 2D shows the correlation between the grayscale intensity of the imaged device and the density of radio opaque material.

FIG. 2E shows a rendering of an exemplary device including a pattern of radio opaque material as positioned in a patient's lung and partially occluded.

FIG. 2F shows a chart of a first exemplary pattern of radio opaque material on an exemplary device.

FIG. 2G shows a rendering of an exemplary device including a pattern of radio opaque material as positioned in a patient's lung and partially occluded, the device having radio opaque material of a density as shown in FIG. 2A.

FIG. 2H shows exemplary rings of radio opaque material of varying size and varying spacing along the length of an exemplary device.

FIG. 2I shows a chart of a second exemplary pattern of radio opaque material on an exemplary device.

FIG. 3A shows an exemplary device including an applicator, a catheter, and a guide wire, the device being shown disassembled.

FIG. 3B shows the applicator of FIG. 3A in an extended position.

FIG. 3C shows the applicator of FIG. 3A in a retracted position.

FIG. 4A shows the device of FIG. 3A, the device being shown assembled.

FIG. 4B shows the device of FIG. 4A, the device being shown with a guide wire extended.

FIG. 5 shows an exploded view of the applicator shown in FIG. 3A.

FIG. 6A shows the applicator of FIG. 3A, a trigger of the applicator being shown in an unlocked position.

FIG. 6B shows the applicator of FIG. 3A, a trigger of the applicator being shown in a locked position.

FIG. 7A shows a partial sectional view of the applicator shown in FIG. 6A.

FIG. 7B shows a partial sectional view of the applicator shown in FIG. 6B.

FIG. 8A shows a portion of the applicator of FIG. 3A, the applicator being viewed from the opposite direction from that shown in FIG. 3A.

FIG. 8B shows a partial sectional view of the applicator of FIG. 3A.

FIG. 9A shows the exemplary assembled device of FIG. 4A, the applicator of the device being shown in an extended position and in proximity to disengaged connector portions.

FIG. 9B shows the exemplary assembled device of FIG. 4A, the distal portion of the shaft being shown in proximity to a removable connector portion.

FIG. 10 shows an exploded view of an exemplary shaft of the exemplary applicator of FIG. 3A.

FIG. 11A shows a sectional view of an exemplary wire extraction button of the exemplary applicator of FIG. 3A.

FIG. 11B shows an exploded view of the exemplary wire extraction button of FIG. 11A.

FIG. 12 shows a sheath luer lock entrance of the exemplary applicator of FIG. 3A.

FIG. 13A shows an exemplary luer lock plug that is configured to engage an exemplary connector of the applicator of FIG. 3A.

FIG. 13B shows the exemplary luer lock plug of FIG. 13A engaging the exemplary connector of the applicator of FIG. 3A.

FIG. 13C shows an assembled view of a connector portion with a sealing arrangement.

FIG. 13D shows an exploded view of the connector portion of FIG. 13C.

FIG. 14 shows an exemplary steerable sheath with a steering mechanism, the steerable sheath being shown in a released configuration.

FIG. 15 shows the exemplary steerable sheath of FIG. 14 with a steering mechanism, the steerable sheath being shown in an active configuration.

FIG. 16A shows an exploded view of the exemplary steerable sheath of FIG. 14.

FIG. 16B shows an exploded view of an embodiment of an exemplary steerable sheath and handle connection mechanism.

FIG. 16C shows an exploded view of the exemplary steerable sheath and handle connection mechanism of FIG. 16B from an alternate view perspective.

FIG. 17A shows a section view of the exemplary steerable sheath of FIG. 14, the steerable sheath being shown in an active configuration.

FIG. 17B shows a section view of the exemplary steerable sheath of FIG. 14, the steerable sheath being shown in a released configuration.

FIG. 17C shows a perspective view of the exemplary handle connection mechanism of FIG. 16B, the handle connection mechanism being shown in a released configuration.

FIG. 17D shows a side view of the exemplary handle connection mechanism of FIG. 16B, the handle connection mechanism being shown in a released configuration.

FIG. 17E shows a perspective view of the exemplary handle connection mechanism of FIG. 16B, the handle connection mechanism being shown in an active configuration.

FIG. 17F shows a perspective view of the exemplary handle connection mechanism of FIG. 16B, the handle connection mechanism being shown in an active and unlocked configuration.

FIG. 17G shows a partial cutaway view of the exemplary handle connection mechanism of FIG. 17F.

FIG. 17H shows a perspective view of the exemplary handle connection mechanism of FIG. 16B, the handle connection mechanism being shown in an active and locked configuration.

FIG. 17I shows a partial cutaway view of the exemplary handle connection mechanism of FIG. 17H.

FIG. 18A the exemplary steerable sheath of FIG. 14 at a first stage of a connection process to a handle of an exemplary applicator such as that shown in FIG. 3A.

FIG. 18B shows the exemplary steerable sheath and exemplary handle of FIG. 18A at a second stage of the connection process.

FIG. 18C shows the exemplary steerable sheath and exemplary handle of FIG. 18A at a third stage of the connection process.

FIG. 19A shows the exemplary steerable sheath of FIG. 14 connected to an exemplary applicator such as that shown in FIG. 3A, with a connector element of the applicator shown in a retracted position.

FIG. 19B shows the exemplary steerable sheath and exemplary applicator of FIG. 19A, with the connector element of the applicator shown in an extended position.

FIG. 20 shows a representative anatomical bifurcation.

FIG. 21 shows a flowchart of an iterative process of navigation of an instrument through anatomical cavities, passing a number of bifurcations.

FIG. 22A shows a perspective view of an embodiment of a control portion of a steering mechanism.

FIG. 22B shows an exploded view of the control portion of the steering mechanism of FIG. 22A.

FIG. 22C shows a section view of the control portion of the steering mechanism of FIG. 22A, control portion being positioned in a released configuration.

FIG. 22D shows a section view of the control portion of the steering mechanism of FIG. 22A, control portion being positioned in an active configuration.

FIG. 23 shows a perspective view of an exemplary braid with an exemplary radiopaque element positioned thereon.

### DETAILED DESCRIPTION

The present invention will be further explained with reference to the attached drawings, wherein like structures are referred to by like numerals throughout the several views. The drawings shown are not necessarily to scale, with emphasis instead generally being placed upon illustrating the principles of the present invention. Further, some features may be exaggerated to show details of particular components.

The figures constitute a part of this specification and include illustrative embodiments of the present invention and illustrate various objects and features thereof. Further, the figures are not necessarily to scale, some features may be exaggerated to show details of particular components. In addition, any measurements, specifications and the like shown in the figures are intended to be illustrative, and not restrictive. Therefore, specific structural and functional details disclosed herein are not to be interpreted as limiting, but merely as a representative basis for teaching one skilled in the art to variously employ the present invention.

Among those benefits and improvements that have been disclosed, other objects and advantages of this invention will become apparent from the following description taken in conjunction with the accompanying figures. Detailed embodiments of the present invention are disclosed herein; however, it is to be understood that the disclosed embodiments are merely illustrative of the invention that may be embodied in various forms. In addition, each of the examples given in connection with the various embodiments of the invention which are intended to be illustrative, and not restrictive.

Throughout the specification and claims, the following terms take the meanings explicitly associated herein, unless the context clearly dictates otherwise. The phrases “in one embodiment” and “in some embodiments” as used herein do not necessarily refer to the same embodiment(s), though it may. Furthermore, the phrases “in another embodiment” and “in some other embodiments” as used herein do not necessarily refer to a different embodiment, although it may. Thus, as described below, various embodiments of the invention may be readily combined, without departing from the scope or spirit of the invention.

The term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

As used herein, the term “radio opaque” refers to a material that is characterized in that electromagnetic radiation (including, but not limited, to X-rays) is unable to pass through such a material.

In some embodiments, the present invention is a device, comprising:

an applicator;
a shaft;
a catheter;
a guide wire;
a connector;
a handle;
a trigger;
a luer lock plug; and
a radio opaque material;
wherein the applicator has an inner open channel from a proximal end to a distal end of the applicator,
wherein the inner open channel of the applicator is of a sufficient size to house the shaft;
wherein the shaft is of a sufficient size to house the catheter and the guide wire,
wherein the catheter and the guide wire are configured to have an extraction button which allow the guide wire to protrude out the catheter,
wherein the catheter and the guide wire are configured to have pre-curved distal tip
wherein the catheter proximal end is configured to have a luer lock entrance
wherein the guide wire is configured to be connected or detached from the catheter,
wherein the shaft is configured allow displacement inside and outside the applicator,
wherein the shaft distal end is configured to allow the shaft to rotate,
wherein the shaft distal end is configured to be connected or detached from the connector,
wherein the distal end of the applicator is attached to the connector,
wherein the connector is configured to attach to a bronchoscope,
wherein the connector is configured to be connected or detached from the bronchoscope,
wherein the connector is configured to include a luer lock plug,
wherein the proximal end of the applicator is attached to the handle,
wherein the handle comprises a switch configured to lock and unlock the handle,
wherein the handle is configured to rotate from an open position to a closed position,
wherein the shaft is configured to rotate with the handle, and
wherein the radio opaque material is attached to an outer portion of the device.

In some embodiments, the radio opaque material is dispersed in a pattern.

In some embodiments, the pattern is not uniform.

In some embodiments, the dispersed pattern comprises a plurality of deposited densities of the radio opaque material on the outer portion of the device.

In some embodiments, a first deposited density of the deposited densities is not identical to a second deposited density of the deposited densities.

In some embodiments, the pattern comprises at least one shape.

In some embodiments, the at least one shape can be a ring.

In some embodiments, the ring can be an unbroken ring.

In some embodiments, the ring can be a broken ring.

In some embodiments, the pattern is in a longitudinal conformation in reference to the applicator.

In some embodiments, the grip handle is free to rotate with respect to the shaft. In some embodiments, the grip handle is constrained from rotation with respect to the shaft. In some embodiments, the grip handle is selectively either free to rotate with respect to the shaft or constrained from rotation with respect to the shaft. In some embodiments, the selective freedom or restriction of rotation of the grip handle with respect to the shaft is independent from restriction of longitudinal motion of the shaft.

In some embodiments, the guide wire is curved.

In some embodiments, the catheter is curved.

In some embodiments, the catheter has a pull wire allowing the curvature of the distal end of the catheter to be manipulated.

In some embodiments, the shaft includes a mechanism allowing rotation of the handle to be controlled.

In some embodiments, the device includes a locking mechanism configured to selectively lock or unlock movement of the catheter along a longitudinal axis of the device, while allowing the catheter to rotate about the longitudinal axis.

In some embodiments, the shaft includes a groove that allows the catheter to be inserted along the side of the shaft.

In some embodiments, the device includes a polytetrafluoroethylene tube located inside the shaft so as to hold the catheter and guide the catheter outside the shaft.

In some embodiments, the guide wire can be extracted from the catheter by demand in order to control the effective curvature of the distal tip of the device. In some embodiments, the device includes a manipulator that is configured to control the motion of the guide wire.

In some embodiments, the guide wire can be detached from the catheter.

In some embodiments, the catheter can be detached from the handle.

In some embodiments, the handle is configured to be detached from the connector without firs extracting the catheter and/or the guide wire from the device.

In some embodiments, the connector is configured to allow the device to be detached from the bronchoscope without first extracting the catheter and/or the guide wire from the device

In some embodiments, the connector includes a luer lock plug configured to be positioned therein so as to allow for connection of a slip tip or a luer lock syringe.

In some embodiments, the catheter includes a luer lock entrance configured to be positioned therein so as to allow for connection of a slip tip or a luer lock syringe.

In some embodiments, the catheter can be used without the guide wire.

In some embodiments, the handle has a component configured to provide for data storage and for contactless communication. In some embodiments, the device stores a unique identifier that can be read in a contactless manner (e.g., through radio-frequency identification or near-field communication technology). In some embodiments, the handle includes an electronic device having general computing, data storage and wireless communication abilities. In some embodiments, a unique identifier is stored in the handle. In some embodiments, the handle unique identifier includes unique barcode that can be read by a barcode reader. In some embodiments, the barcode is stamped on the handle. In some embodiments, the barcode is stamped on the handle package. In some embodiments, the barcode is included in a product label.

In some embodiments, a radio opaque material includes, but is not limited to, materials including barium, iodine, or any combination thereof. In some embodiments, two or more radio opaque materials are used in conjunction with one another.

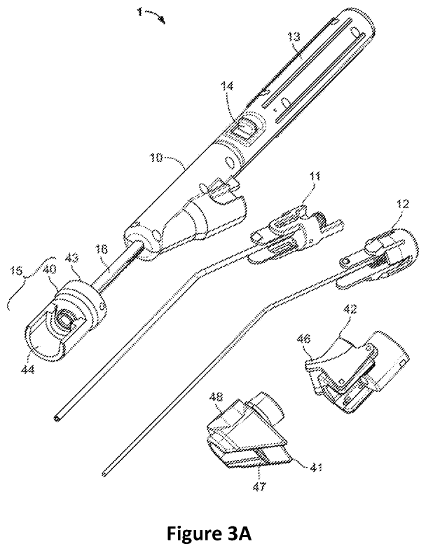

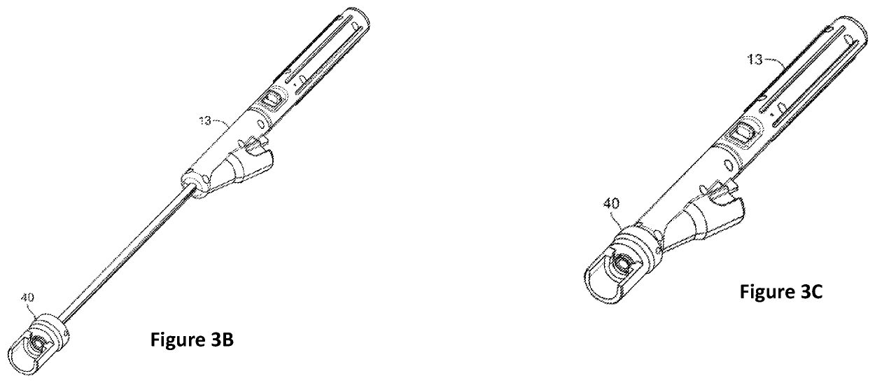

FIG. 3A shows the elements of an exemplary device 1. In some embodiments, the device 1 includes an applicator 10, a catheter 11 and a guide wire 12. In some embodiments, the applicator 10 includes a grip handle 13 that allows the user to pull, push, or rotate the grip handle 13 from a closed (retracted) position to an open (extended) position. In some embodiments, the applicator 10 includes an applicator shaft 16 that allows the grip handle 13 to slide along the applicator shaft 16 (i.e., along a longitudinal axis) while avoiding relative rotation between the applicator shaft 16 and the grip handle 13. In some embodiments, the applicator shaft 16 includes an internal passage that is configured to receive the catheter 11. Consequently, in some embodiments, rotation of grip the handle 13 causes the applicator shaft 16 to rotate therewith. In some embodiments, rotation of the grip handle 13 with respect to the shaft 16 can be selectively locked or unlocked, such that, when unlocked, the grip handle 13 is free to rotate with respect to the shaft 16. In some embodiments, the applicator 10 includes a connector element 15 that enables connection of the applicator 10 to any commercially used bronchoscope. In some embodiments, the connector element 15 includes a connector portion 40 that is permanently connected to the shaft 16. In some embodiments, the connector portion 40 is configured to connect the device 1 to a commercially used bronchoscope. In some embodiments, the connector portion 40 is connected to a bronchoscope by manually rotating swivel ring 43 in one direction, so as to move the swivel ring 43 toward and press a connector coupling 44 against the bronchoscope. In some embodiments, to detach the device 1 from the bronchoscope, the swivel ring 43 is manually rotated in the other direction, thereby moving the swivel ring 43 away from the connector coupling 44 and releasing pressure by the connector coupling 44 on the bronchoscope.

In some embodiments, the connector element 15 includes a connector portion 41 that can be detached from the shaft 16, and a connector portion 42 that can be detached from the shaft 16. In some embodiments, the connector portion 41 and the connector portion 42 may be connected to the shaft 16 by a snap 45 that is located at the distal end 32 of the shaft 16. In some embodiments, the connector portion 41 can be connected to a commercially available bronchoscope by sliding the connector portion 41 over an entrance port of the bronchoscope. In some embodiments, the connector portion 41 includes a connector slider 47 that is configured to slide over the entrance port of the bronchoscope and thereby lock the connector portion 41 to the bronchoscope. In some embodiments, the connector portion 41 includes a release button 48 that is operable to release the connector portion 41 from the bronchoscope. In some embodiments, the connector portion 42 includes a connector clasp 46. In some embodiments, the connector portion 42 can be connected to a commercially available bronchoscope by closing the connector clasp 46 against an entrance port of the bronchoscope. In some embodiments, the connector portion 42 can be removed from a commercially available bronchoscope by opening the connector clasp 46. In some embodiments, the connector portion 41 and the connector portion 42 can be connected to a bronchoscope in the absence of the applicator 10.

In some embodiments, the grip handle 13 includes a trigger 14 that is configured to lock the grip handle 13 at any position along its travel between its open and closed positions (e.g., along the applicator shaft 16). In some embodiments, the distal end of the shaft 16 is configured to act as a swivel, allowing the shaft 16 and the grip handle 13 to rotate with respect to the connector element 15 along the longitudinal axis to any desired angle.

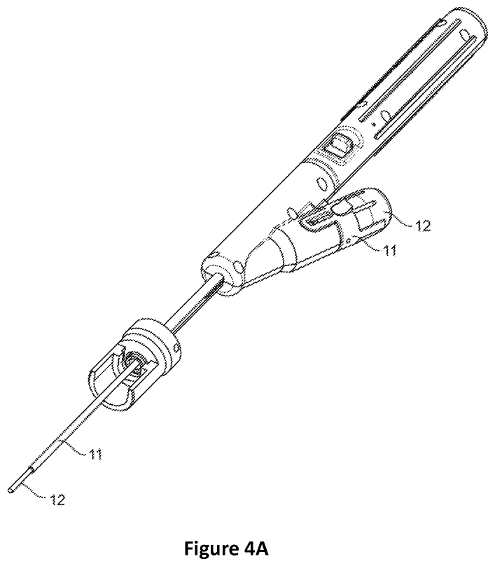

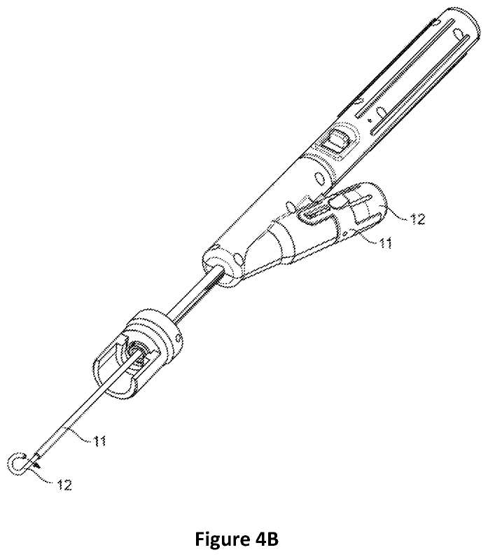

FIG. 3B shows the device 1 of FIG. 3A in its open (extended) position. The connector element 15 is extended distally from the grip handle 13. FIG. 3C shows the device 1 from FIG. 3B in its closed (retracted) position. The connector element 15 is in its closest proximity to the grip handle 13. FIG. 4A shows the device 1 of FIG. 3A, as configured with both the catheter 11 and the guide wire 12 connected to grip handle 13. FIG. 4B shows the device 1 of FIG. 4A, but with the guide wire 12 extended. In some embodiments, the device 1 includes a wire extraction button 33, which is configured to allow the guide wire 12 to be extended. In some embodiments, as shown in FIG. 4B, the guide wire 12 is flexible and can be positioned as needed.

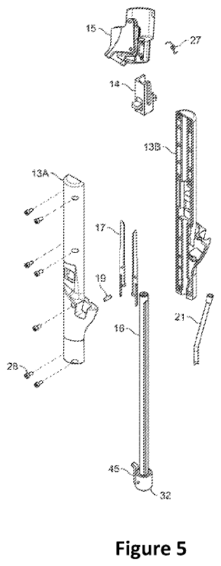

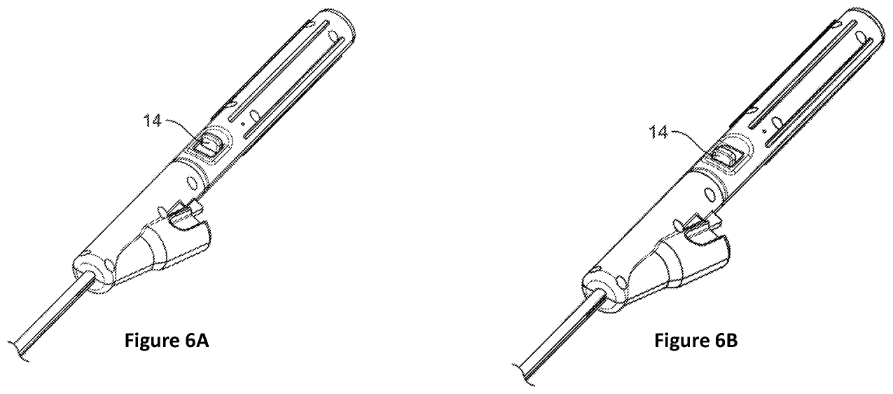

FIG. 5 shows an exploded view of the applicator 10. The grip handle 13 is divided into two side portions 13A and 13B. Screws 28 are configured to connect the two side portions 13A and 13B to one another. The applicator 10 includes a trigger 14, a lever 17, a hinge 19, and a spring 27, which will be described in detail with reference to FIGS. 6A and 6B below. The applicator 10 also includes an inlet tube 21 that is configured to receive the catheter 11.

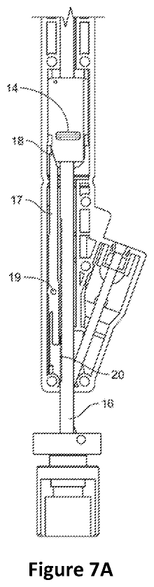

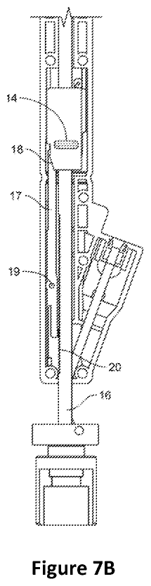

FIG. 6A shows the device 1 with the trigger 14 in its unlocked position, in which the shaft 16 is allowed to move with respect to the grip handle 13. FIG. 6B shows the device 10 with the trigger 14 in its locked position, in which the shaft 16 is allowed to move with respect to the grip handle 10. FIG. 7A shows a sectional view of the device 1 with the trigger 14 in its unlocked position. FIG. 7B shows a sectional view of the device 10 with the trigger 14 in its locked position. The device 1 includes a lock lever 17 that is pivotably engaged with a hinge 19. The shaft 16 has a grooved portion 20. The trigger 14 has an angled surface 18 that is configured to engage the lock lever 17 when the trigger 14 is in its locked position, and to disengage the lock lever 17 when the trigger 14 is in its unlocked position. When the angled surface 18 of the trigger 14 engages the lock lever 17 (e.g., as shown in FIG. 7B), the lock lever 17 pivots about the hinge 19 to a position such that the lock lever 17 engages the grooved portion 20 of the shaft 16, thereby preventing the shaft 16 from axial motion with respect to the grip handle 13. Conversely, when the angled surface 18 of the trigger 14 disengages the lock lever 17 (e.g., as shown in FIG. 7A), the lock lever pivots about the hinge 19 to a position such that the lock lever 17 does not engage the grooved portion 20 of the shaft 16, thereby allowing the shaft 16 to move axially with respect to the grip handle 13.

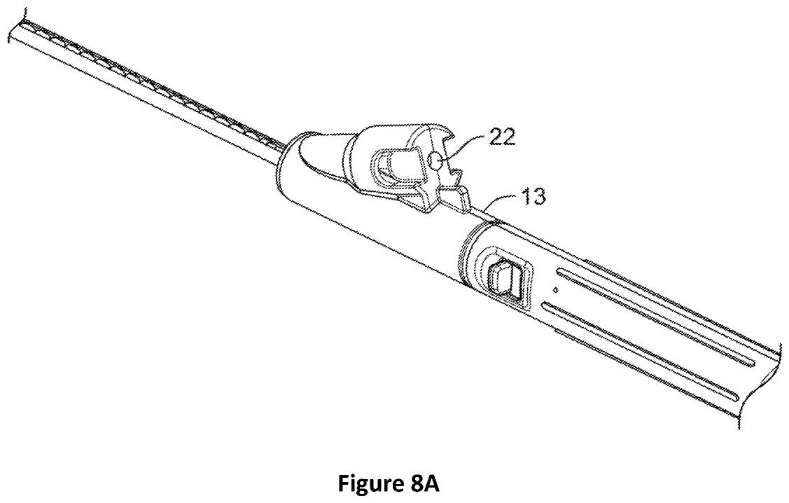

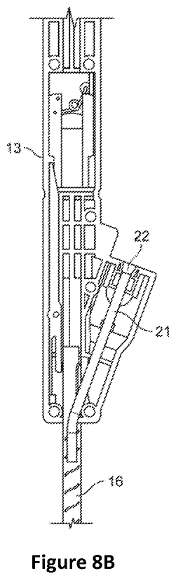

FIG. 8A shows a perspective view of the grip handle 13 in a direction facing toward the distal end of the grip handle 13. The grip handle 13 includes an inlet port 22 that allows insertion of the catheter 11 into the applicator 10. FIG. 8B shows a sectional view of a portion of the grip handle 13. The grip handle 13 includes an inlet tube 21 extending from inlet port 22 to the internal passage of the shaft 16, and configured to allow passage of the catheter 11.

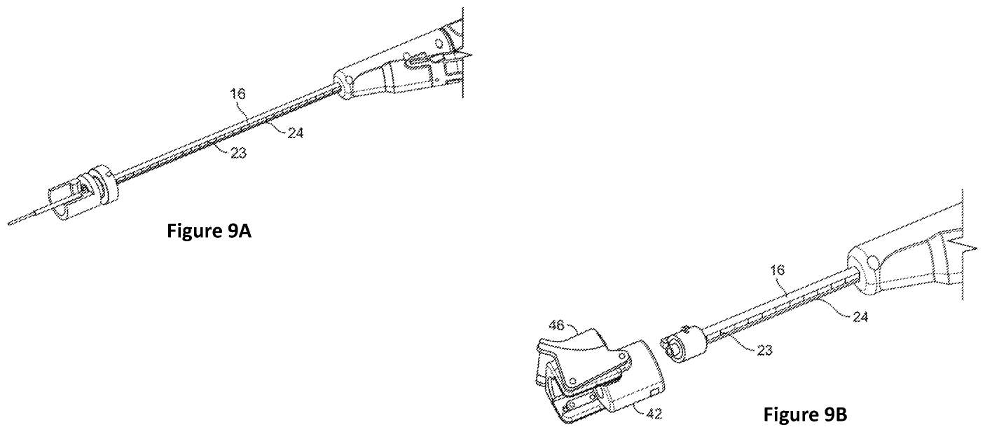

FIG. 9A and FIG. 9B show an opening 24 along the shaft 16 that allows the inlet tube 21 to slide from its extended position (i.e., as shown in FIG. 3B) to its closed position (i.e., as shown in FIG. 3C). In some embodiments, in order to prevent the catheter 11 and the guide wire 12 from buckling and protruding from the shaft 16 due to friction in a bronchoscope that is connected to the device 1, a polytetrafluoroethylene (“PTFE”, such as the material sold under the trade name TEFLON by DuPont) tube 23 is positioned inside the shaft 16 to act as a flexible barrier. In some embodiments, the PTFE tube 23 is positioned around the shaft 16 rather than inside the shaft 16. In some embodiments, rather than a PTFE tube 23, a spring, telescoping material or other flexible material that can withstand the buckling force is used. FIG. 9A shows the PTFE tube 23 in an extended position. FIG. 9B shows the PTFE tube 23 in a compressed position. In some embodiments, the PTFE tube 23 is connected to the connector element 15 at the distal end of the PTFE tube 23 and to the inlet tube 21 at the proximal end of the PTFE tube 23. As shown in FIG. 9B, when the connector element 15 is positioned proximate to the grip handle 13, the PTFE tube 23 is compressed.

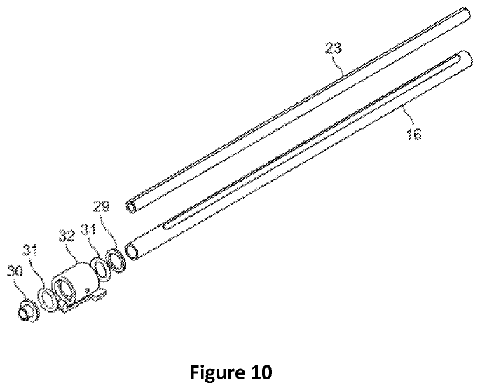

FIG. 10 shows an exploded view of the shaft 16. In some embodiments, the shaft 16 includes a swivel mechanism. In some embodiments, a PTFE tube 23 is positioned within the shaft 16 to act as a flexible barrier. In some embodiments, a shaft distal end 32 is free to rotate with respect to the shaft 16. In some embodiments, the swivel mechanism also includes two washers 29 and 30 and two o-rings 31 that provide control to the rotation. In some embodiments, the shaft distal end 32 is configured to be attached to the connector 15.

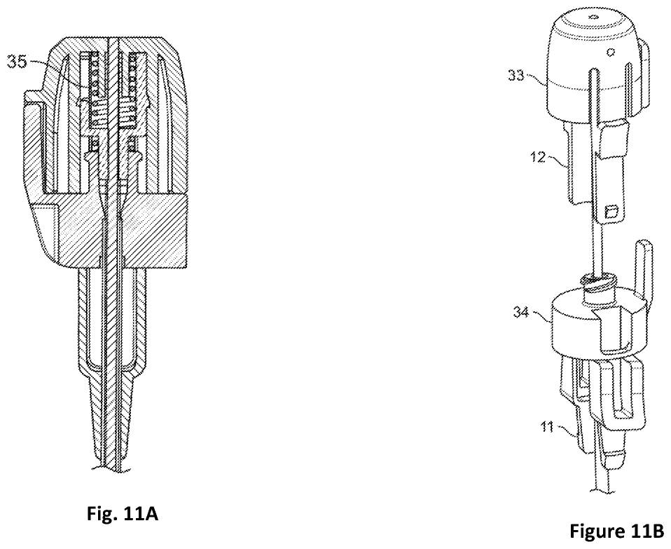

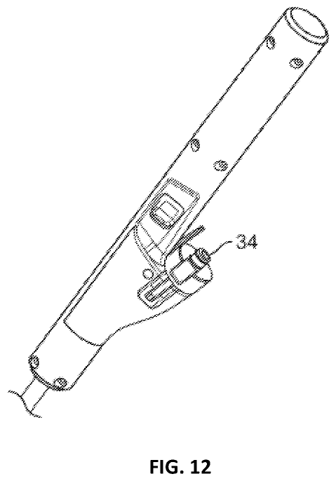

FIG. 11A and FIG. 11B show a sectional view and an exploded view, respectively, of a wire extraction button 33. In some embodiments, the wire extraction button 33 presses against a spring 35, which biases the wire extraction button 33 to a position in which the wire extraction button 33 restrains movement of the guide wire 12. In some embodiments, the wire extraction button 33 is removably coupled to a sheath luer lock entrance 34, which is configured to allow connection to a syringe. In some embodiments, the wire extraction button 33 can be removed to expose the sheath luer lock entrance 34. FIG. 12 shows the proximal portion of the applicator 10 with the sheath luer lock entrance 34 exposed.

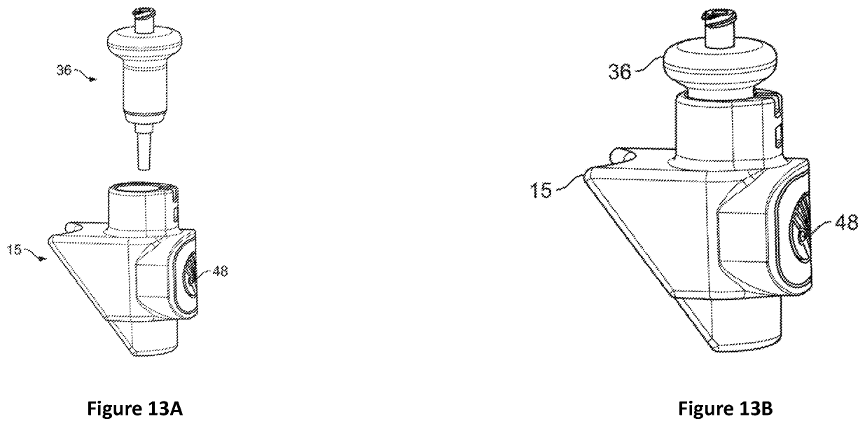

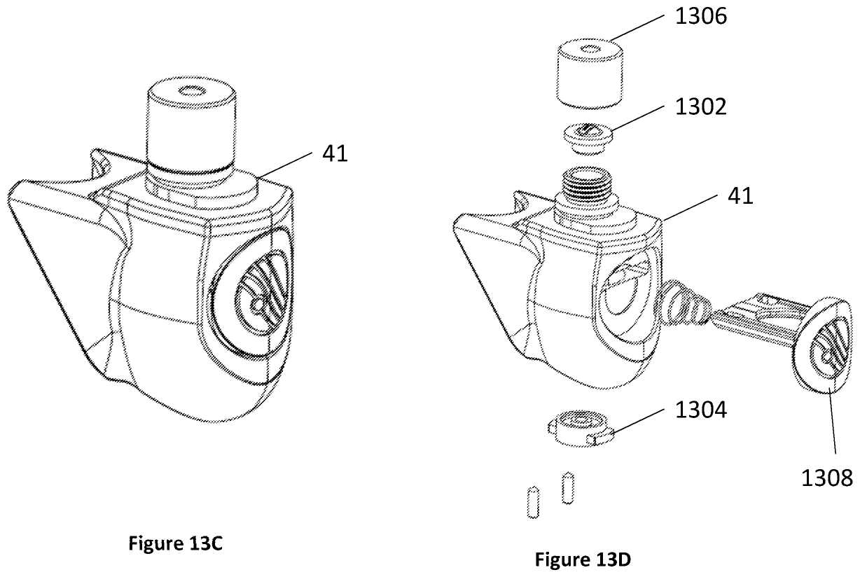

FIG. 13A shows a luer lock plug 36, which can be connected to the connector portion 41 or the connector portion 42 to allow a syringe connection to the connector 15. FIG. 13B shows the luer lock plug 36 as connected to the connector 15.

In some embodiments, the connector portion 41 includes an integrated sealing arrangement. FIG. 13C and FIG. 13D show an assembled view and an exploded view, respectively, of a connector portion 41 with an integrated sealing arrangement. In some embodiments, the sealing arrangement includes an upper seal 1302 and a lower seal 1304. In some embodiments, the upper seal 1302 is configured to allow a user to perform suction of fluids or injection of fluids when no catheter is present. In some embodiments, the lower seal 1304 is configured to provide a fluid-tight seal between the connector portion 41 and a bronchoscope. In some embodiments, the sealing arrangement includes a sealing cap 1306 configured to cover the upper seal 1302. In some embodiments, connector portion 41 includes a release button 1308 operable to release the connector portion 41 from a bronchoscope.

In some embodiments, the present invention relates to a radio opaque pattern on a device, where the radio opaque pattern can be visualized by a user (e.g., a doctor, etc.) and used to identify the specific portion of the device visible on the x-ray image, e.g., by correlating portions of the device with the observed density of the radio opaque material. In some embodiments, the radio opaque material is positioned on the catheter 11 of the device 1. In some embodiments, the radio opaque material is positioned on the guide wire 12 of the device 1. In some embodiments, the radio opaque material is positioned on both the catheter 11 and the guide wire 12 of the device 1, which cooperate to produce a combined “effective” pattern of radio opaque material on the device 1.

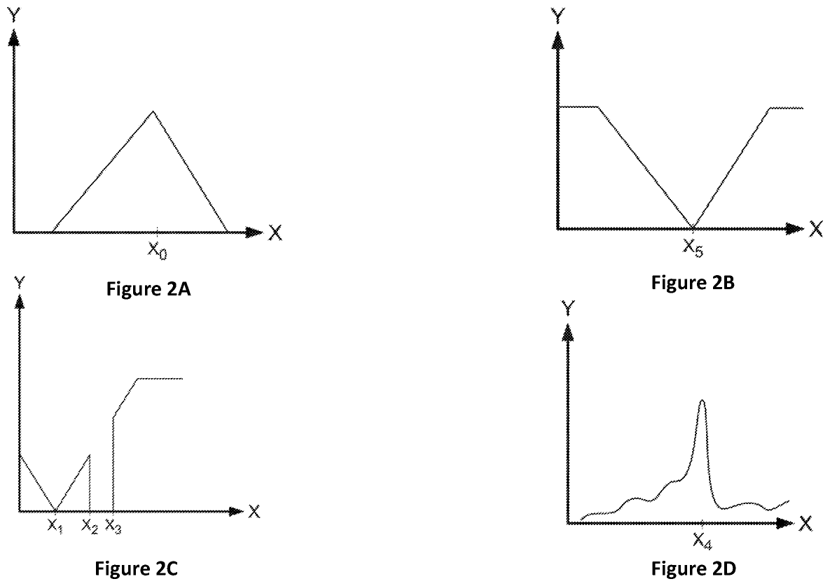

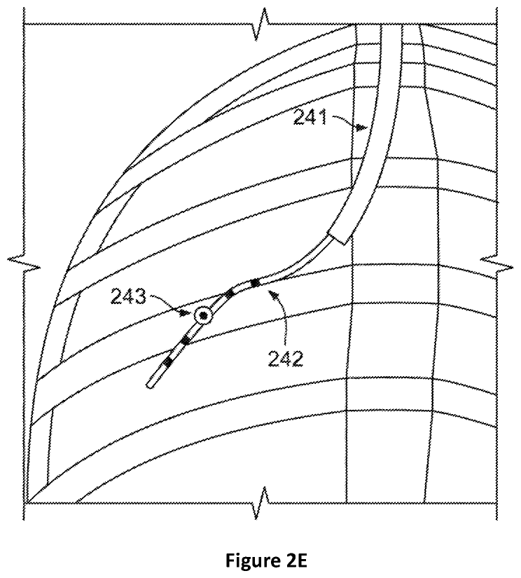

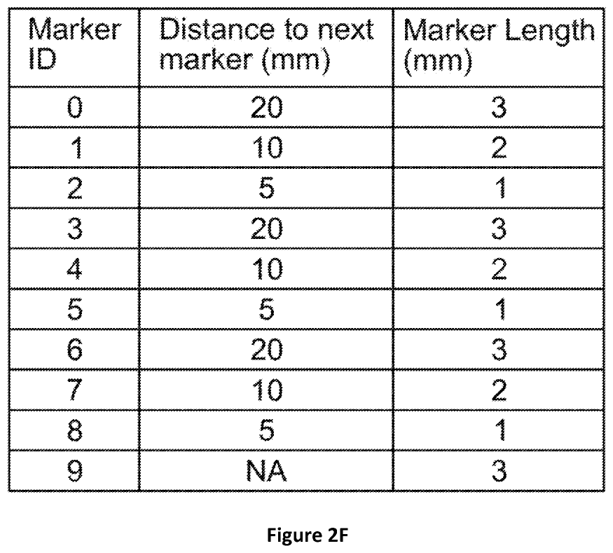

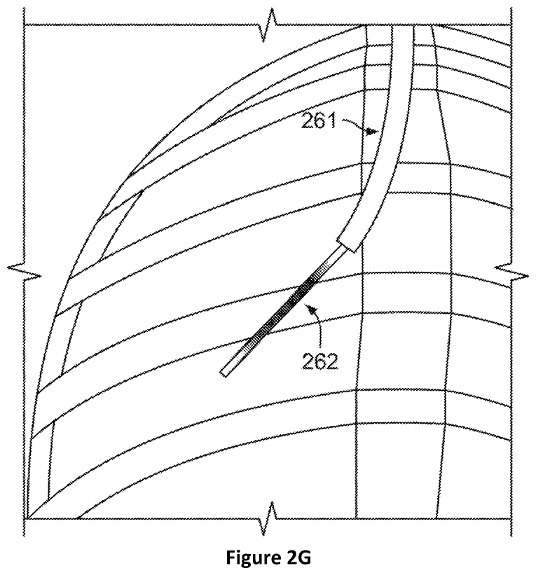

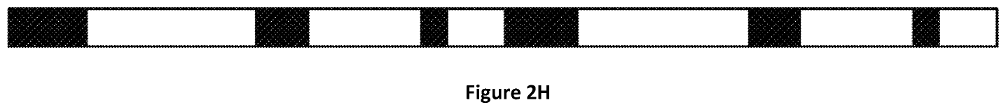

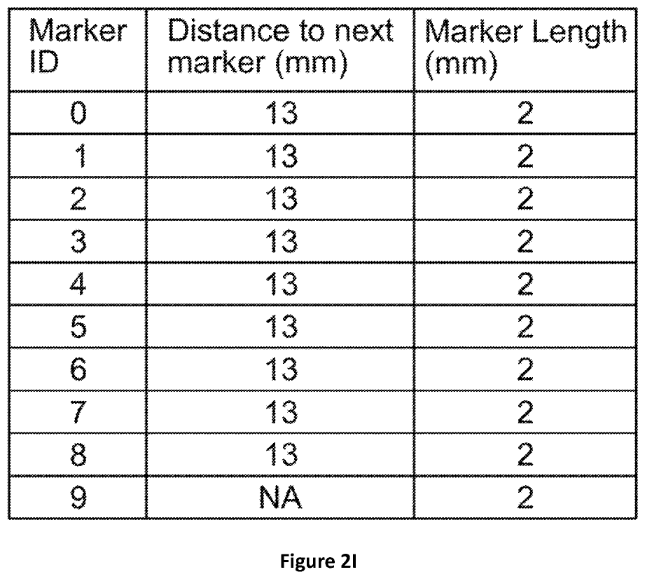

In some embodiments, the device 1 of the current invention has a radio opaque material positioned in a pattern which can be observed (e.g., but not limited to, using X-ray images of the device), where the pattern has been manufactured by applying variable amount(s) of radio opaque material along the device. In some embodiments, the correlation between the function of radio opaque material density along the device and the function of grayscale intensity in the x-ray image allows the detection of a specific portion of the device on the fluoroscopic image in spite of partial occlusion by other radio opaque objects on the image. In some embodiments, the higher density of radio opaque material in the device results in lower gray-scale intensities visualized by the X-ray image and vice versa. FIG. 2A shows a plot of radio opaque material density along the length of an embodiment of device (Y axis), as plotted against the length of the device (X axis). FIG. 2B shows one-dimensional gray scale levels (Y axis) of a device with material density as shown in FIG. 2A, as imaged by a fluoroscope along the length of the device (X axis). Taken together, FIGS. 2A and 2B show that the density of the radio-opaque material is correlated with gray-scale image function.

FIG. 2C shows one-dimensional gray scale levels (Y axis) of a partial device protruding from a bronchoscope (as compared to FIG. 2B, which illustrates the full image of the device), imaged by a fluoroscope along to the length of the device (X axis). The zero value between positions x2 and x3 along the X axis illustrates an occlusion that blocks the X-ray radiation in this interval. FIG. 2D shows the absolute value of the correlation function between the partially imaged device (i.e., as shown in FIG. 2C) and the density of the radio opaque material (i.e., as shown in FIG. 2A). The position of the peak in FIG. 2D can be utilized to calculate the translation between pixels in FIG. 2C and 3 dimensional model coordinates in FIG. 2A. FIG. 2E shows a representation of an X-Ray image showing a bronchoscope 241 and device 242 (e.g., the device 1) with radio opaque material, as positioned within the chest of a patient. At position 243, the device 242 is occluded by an ECG patch.

In some embodiments, the radio opaque material is arranged along the device 1 in a pattern. In some embodiments, the pattern includes differently sized rings extending around the device. In some embodiments, the pattern includes rings irregularly spaced along the device. FIG. 2F shows a table showing a first pattern comprised of rings of radio opaque material located at different spacing from one another and having different lengths. FIG. 2I shows a table showing a second pattern comprised of rings of radio opaque material located at different spacing from one another and having different lengths. It will be apparent to those of skill in the art that the specific patterns represented by FIG. 2F and FIG. 2I are only exemplary and that other patterns are possible.

FIG. 2G shows a representation of an X-Ray image showing a bronchoscope 261 and device 262 (e.g., the device 1) having radio opaque material that is patterned as shown in FIG. 2A. FIG. 2H shows an illustration of a pattern of radio opaque material containing rings of variable size, placed in positions at varying intervals along the outer portion of a device (e.g., the device 1).

In a non-limiting example, when a portion of a pattern of radio opaque material is visible, a user can calculate the one-dimensional translation (e.g., correlation) between the imaged pattern and the density function. The relation between the radio opacity of the device and the gray-scale levels can be used for this purpose. In another non-limiting example, a user can use a template matching method that searches for the highest correlation between the gray-scale levels of the visible segment of the device in the image and the radio opaque density profile of the device. Such a method is robust to occlusion and noise caused by objects that are behind or above the device with respect to the projection direction from an X-ray tube to an image intensifier. In some embodiments, FIG. 2D shows an exemplary correlation function between the device's partial image as shown in FIG. 2C and the device's pattern of radio opaque material density as shown in FIG. 2A. For instance, the translation between the density function at point x0 in FIG. 2A to the pixel gray-scale level at point x1 on FIG. 2C corresponds to the peak position at the point x4 in the correlation function shown in FIG. 2D. As a result, although the device as represented by FIG. 2C is partially visible and partially occluded in the area between points x2 and x3, it is possible to perform device localization on the image and correlate each pixel of the visible device, as represented by FIG. 2D to the known model for the device, as represented by FIG. 2A.

In some embodiments, a unique radio opaque pattern is manufactured through attaching radio opaque rings of variable size to the device at specific positions along the device's longitude direction axis, as illustrated by FIG. 2H. The unique radio opaque pattern assists a user in estimating the transformation function between the imaged device's pixels and predesigned device model for manufacturing. This transformation function can be estimated by finding a function that satisfies the constraints imposed by the different marker sizes and locations on the device. A non-limiting example for such design, which is robust to occlusion of several markers on x-ray image, is provided in FIG. 2F.

In some embodiments, a medical image (e.g., an X-ray image) of at least a portion of a body of patient with the device 1 (i.e., which includes the radio opaque material) positioned within the body of the patient can be analyzed to determine the depth of the device 1 within the body based on knowledge of the positioning of the radio opaque material. In some embodiments, the current invention relates to a method to recover 3-dimensional depth information in such cases, where due to occlusions and noise of the 2-dimensional image as an input, such as X-ray image or video image sequence, some markers may not be detected, by means of unique pattern on the device as shown, for example, in FIG. 2A. The occlusion and noise of the input image or video image sequence may be caused by occlusion of medical devices, high density tissue such as ribs, patient pace makers, ECG cables, etc. as illustrated by FIG. 2E.

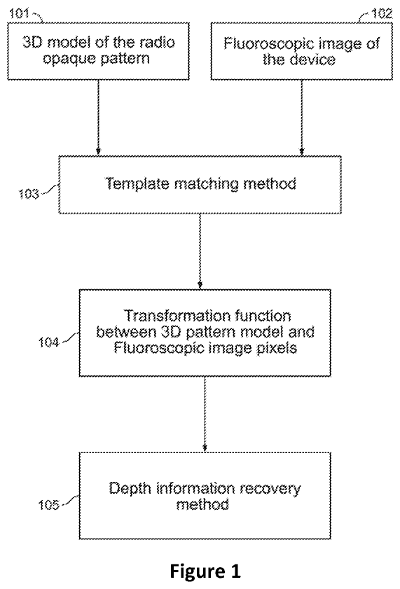

FIG. 1 shows a flowchart of a process for determining the depth of an exemplary device (e.g., the device 1 of FIG. 3A). The process receives, as inputs, a density model (101) of the radio opaque material along the device (e.g., the information shown in FIG. 2A) and fluoroscopic image data (102) showing the device positioned within the patient's body. A transformation function (104) between the model and the image pixels is calculated using a template matching method (103). In some embodiments, the template matching method is performed as described above with reference to FIGS. 2A-2D. The transformation function is used for depth information recovery (105).

In some embodiments, the depth of the device can be calculated from a single image based on prior knowledge the physical dimensions of the specific radio opaque pattern. For instance, given the known physical distance between two points that are identified and located in the intra operative image, one can determine the relative depth between these two points. In some embodiments, such a technique for determining relative depth is carried out as described in International Patent Application Publication No. WO/2015/101948, the contents of which are incorporated herein by reference in their entirety. More particularly, in some embodiments, a device (e.g., the device 1) or a portion thereof (e.g., the portion between two of the stripes shown in FIG. 2H) having a known length “L3” and located in three-dimensional space within a patient's body is projected into an imaging plane to create a projection image including such a device. The observed (i.e., projected) length of the same device (or device portion) in the two-dimensional imaging plane is “L2”. As shown in FIG. 12 of International Patent Application Publication No. WO/2015/101948, an angle α of the device (or device portion) in space can be determined by solving the equation L2=L3 cos α. The relative depth D between the two ends can then be determined by calculating D=L3 sin α.

In some embodiments, the depth of the device can be calculated using the methods described in International Patent Application Publication No. WO/2017/153839, the contents of which are incorporated herein by reference in their entirety. In some embodiments, such determination is performed according to the following process. In some embodiments, the device is imaged by an intraoperative device and projected to an imaging plane. In some embodiments, a predefined distance “m” between two radiopaque regions “F” and “G” on the device (e.g., two of the stripes shown in FIG. 2H) is considered as an input. In some embodiments, point “F” results from a projection of two possible 3D locations A and B, having different depth from one another. In some embodiments, point “G” results from a projection of two possible depth locations C and D, having different depth from one another, and where C corresponds to A and D corresponds to B. In some embodiments, 3D distances between the back-projected location pairs AC and BD are measured. In some embodiments, the 3D distances AC and BD are compared to the distance “m”, and either points A and C or points B and D are selected based on the best fit. In some embodiments, the depth is that corresponding to the selected pair of locations.

In some embodiments, the depth recovery can be performed using a combination of a known patient anatomy and pose estimation approach. In some embodiments, the knowledge of the unique radio opaque pattern can be combined with the knowledge of the patient's anatomical bronchial tree (e.g., as extracted from the pre-operative image) and the knowledge of the current pose of the imaging device relative to the patient (e.g., a point of view that allows projecting 3D information from a pre-operative image to the current image acquired from the imaging device). Since an instrument is located inside a discrete anatomical space, the current pose estimation information can be used to limit the possible solutions. Furthermore, the matching between the instrument location and possible anatomical location on the bronchial tree can be recovered by solving an optimization problem with respect to the following parameters: an assumption of the anatomical location of the tool, a pose estimation, and potential 3d anatomy changes. In some embodiments, such an approach is described in greater detail in International Patent Application Publication No. WO2015/101948.

In some embodiments, the depth estimation can be performed from a sequence of two or more images by (a) finding corresponding points between views, for example, by tracking or matching by visual similarity; (b) finding pose relative differences using, for example, a jig, human anatomy, or any other pose estimation algorithm (e.g., those described in International Patent Application Publication No. WO/2017/153839); and (c) reconstructing three-dimensional information of the matching points from multiple images with known poses using methods that are known in the art (e.g., triangulation, a stereo corresponding point based technique, a non-stereo corresponding contour method, a surface rendering technique, etc.).

In some embodiments, the device provides increased maneuverability inside a body cavity, e.g., but not limited to, bronchial airways, compared to typical methods. In some embodiments, the device is as seen in the non-limiting example shown in FIGS. 3A-13B. In some embodiments, the exemplary device allows increased accuracy while navigating with one hand and supports the standard diagnostic and therapeutic device's entrance from the other. In some embodiments, the guide wire is pre-curved. In some embodiments, the catheter is pre-curved. In some embodiments, both the guide wire and the catheter are pre-curved. In some embodiments, the guide wire is straight. In some embodiments, the catheter is straight. In some embodiments, both the guide wire and the catheter are straight. In some embodiments, the guide wire is configured to be bent as needed. In some embodiments, the catheter is configured to be bent as needed. In some embodiments, both the guide wire and the catheter are configured to be bent as needed. In some embodiments, the guide wire is configured to protrude past the tip of the catheter, while adding extra bending to the device. This feature allows for increased maneuverability of the device during the navigation inside the lung.

In some embodiments, the device including the radio opaque material includes an endoscope, an endo-bronchial tool, and/or a robotic arm.

In some embodiments, the catheter has a steerable sheath configured to guide an object (e.g., endo-therapy accessories, an ultrasound probe, etc.) to a target area. In some embodiments, the target area is within the respiratory system. In some embodiments, the steerable sheath includes a mechanism that is configured to allow a user to steer and control the distal end of sheath (e.g., the end that is positioned within the body). In some embodiments, the steerable sheath is configured to have a mechanism that allows the distal end of sheath to be locked in a desired position. In some embodiments, the steerable sheath includes one or more radiopaque markers along the length of the sheath. In some embodiments, the radiopaque markers allow the location of the sheath within the patient's body to be determined and/or shown on an augmented image. In some embodiments, the radiopaque markers are positioned in a predesigned pattern of radiopaque markers along the sheath.

In some embodiments, the sheath includes a mechanism that is configured to allow the sheath to be attached to and detached from a handle. In some embodiments, the sheath is sufficiently sized to allow objects (e.g., endo-therapy accessories, an ultrasound probe, etc.) to be introduced therein. In some embodiments, the sheath is sufficiently sized to be introduced through a standard bronchoscope. In some embodiments, the sheath has a luer lock mechanism to allow a syringe connection to allow injection and suction of fluids. In some embodiments, the sheath is configured for multiple uses on a single patient.

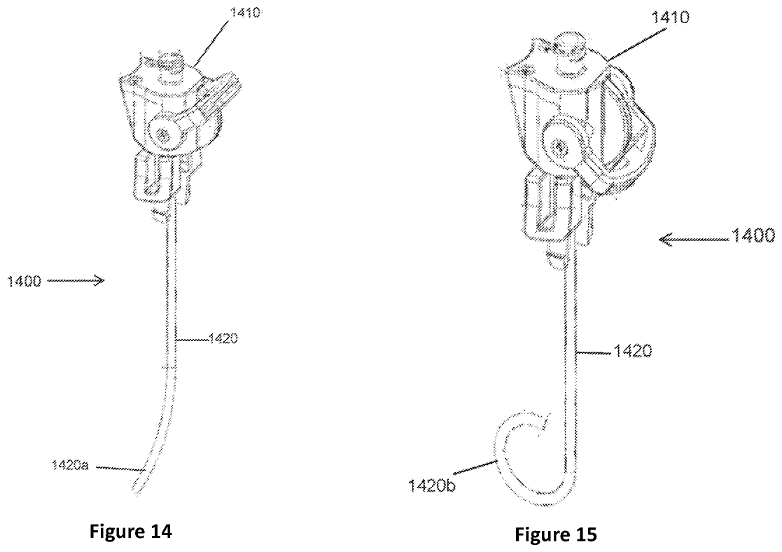

FIGS. 14 and 15 shows perspective views of an exemplary device 1400 including a control portion 1410 and a steerable sheath 1420. FIG. 14 shows the steerable sheath 1420 positioned in a released configuration 1420a. FIG. 15 shows the steerable sheath 1420 positioned in an active configuration 1420b.

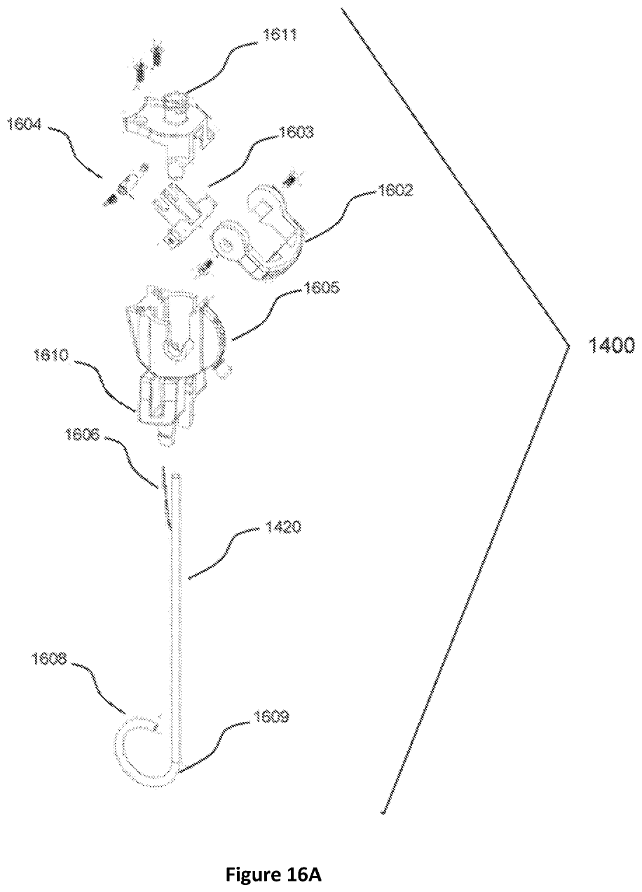

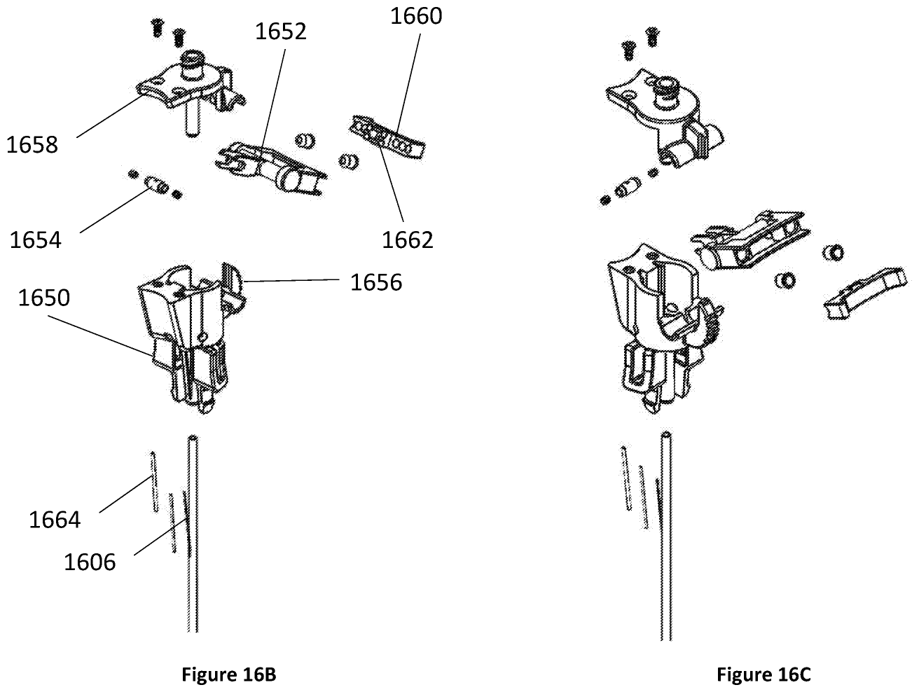

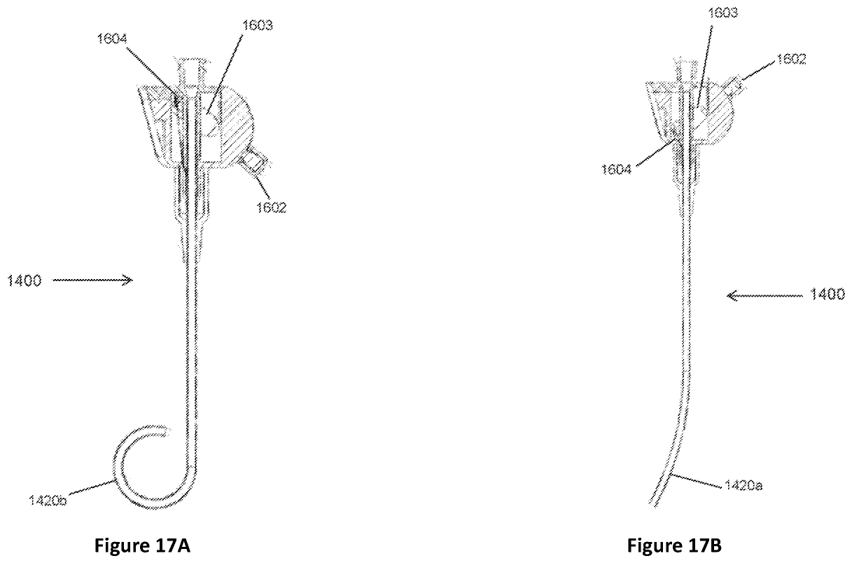

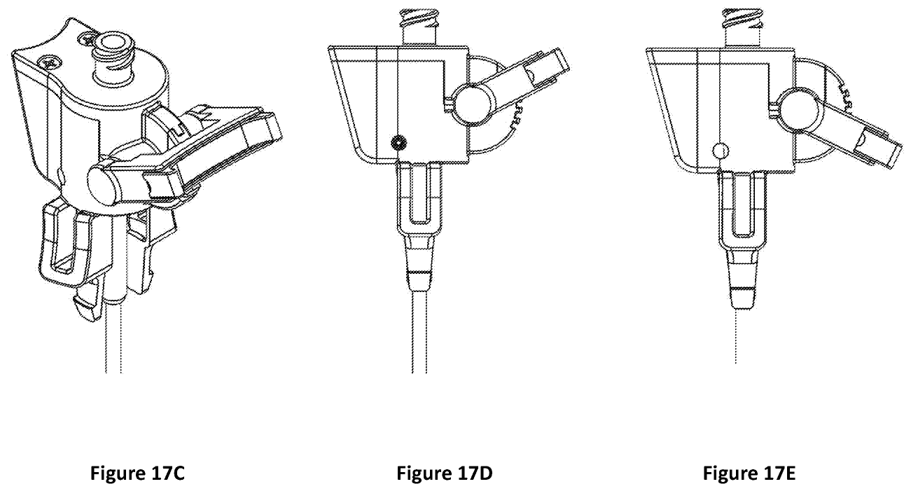

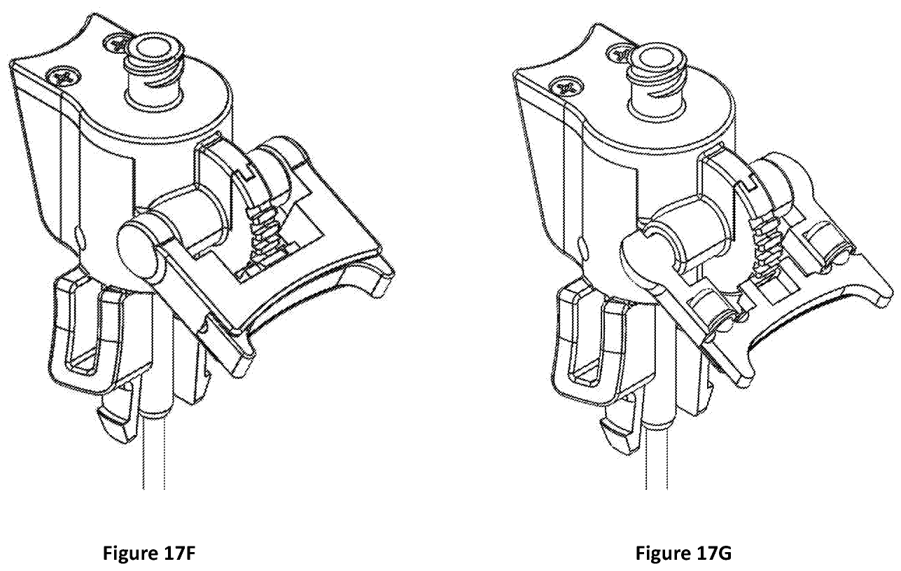

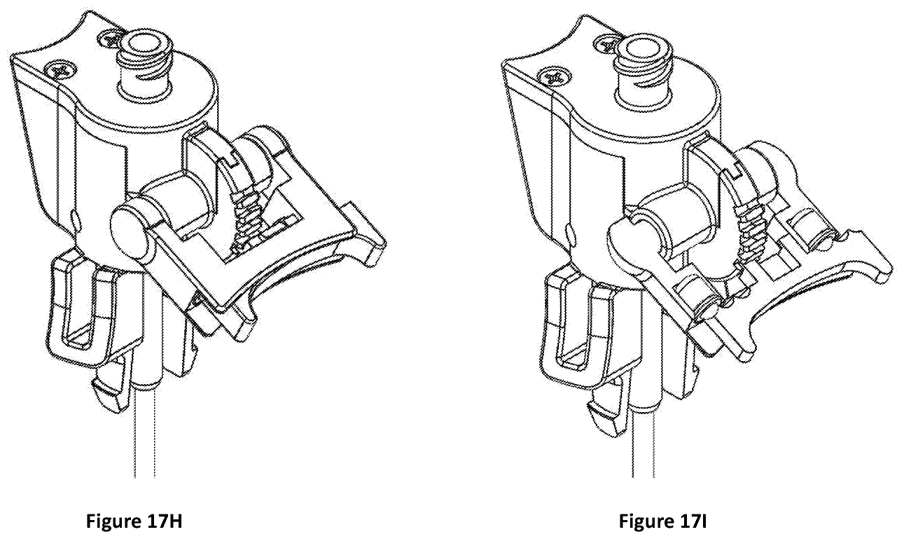

FIG. 16A shows an exploded view of the exemplary device 1400 of FIGS. 14 and 15. FIGS. 17A and 17B show section views of the exemplary device 1400 of FIGS. 14 and 15 in an active configuration and a released configuration, respectively. In some embodiments, the steerable sheath 1420 includes a pull wire 1606 extending through the steerable sheath 1420 to the distal end 1608 of the steerable sheath 1420. In some embodiments, the pull wire 1606 allows manipulation of the curvature of the distal end 1608 of the steerable sheath 1607.

In some embodiments, the control portion 1410 includes a steering mechanism housed within a housing 1601. In some embodiments, the steering mechanism includes a steering lever 1602, a steering shaft 1603, a pull wire locking shaft 1604 positioned within the steering shaft 1603, and a locking knob 1605. In some embodiment, an end of the pull wire 1606 opposite the distal end 1608 of the steerable sheath 1420 is secured to the pull wire locking shaft 1604. In some embodiments, movement of the steering lever 1602 (e.g., between the position shown in FIG. 14 and the position shown in FIG. 15, causes the steering shaft 1603 to move within the housing 1601, causing corresponding motion of the pull wire locking shaft 1604 and of the pull wire 1606. In some embodiments, the steering mechanism is configured such that the steering lever 1602 can be moved so as to deflect or straighten the distal end of the steerable sheath 1420 to a desired angle, thereby allowing the curvature of the distal end 1608 of the steerable sheath 1420 to be manipulated.

In some embodiments, the control portion 1410 also includes a locking mechanism. In some embodiments, a locking knob 1605 with locking cavities is formed on the housing 1601. In some embodiments, the steering lever 1602 can be selectively locked to the locking cavities of the locking knob 1605, thereby locking or unlocking movement of the distal end 1608 of the steerable sheath 1420, while allowing the steerable sheath 1420 to be rotated about its longitudinal axis.

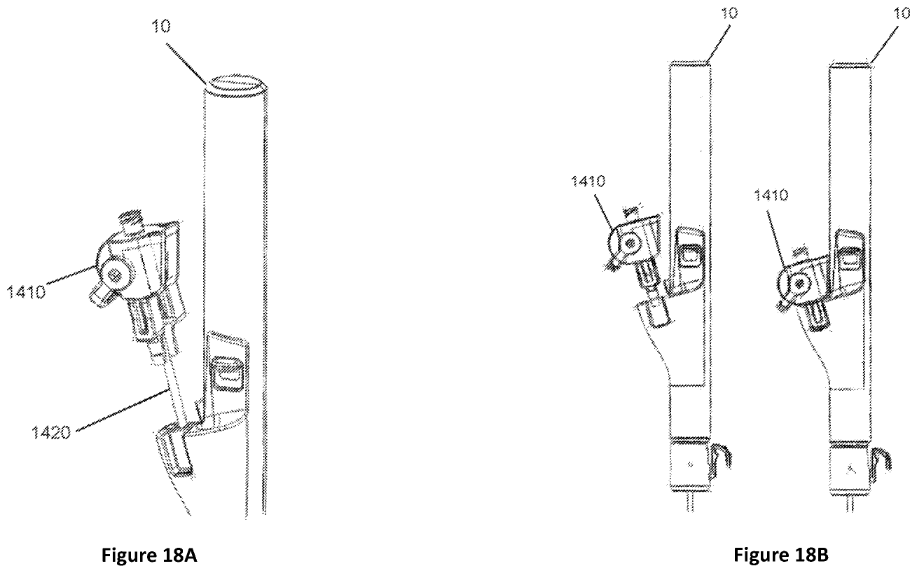

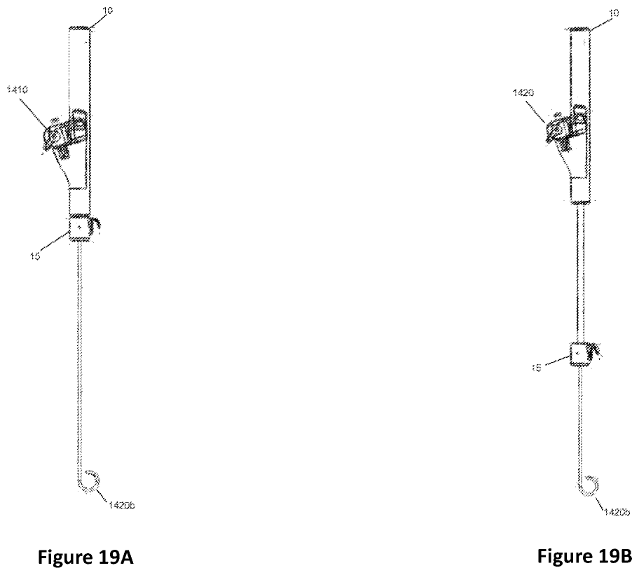

In some embodiments, the device 1400 includes a handle connection mechanism 1610 configured to allow the sheath to be connected to or disconnected from a handle (e.g., the handle of the applicator 10 shown in FIG. 3A). FIG. 18 shows sequential views of the device 1400 being connected to the handle of the applicator 10 shown in FIG. 3A. FIG. 19 shows the device 1400 as connected to the handle of the applicator 10 shown in FIG. 3A, with the connector element 15 positioned either in a retracted position or an extended position.

In some, embodiments the steerable sheath 1420 has a wall that is sufficiently thin so as to allow the sheath to be introduced into a standard bronchoscope having a working channel with a diameter of 2.8 mm and to be able to receive therewithin and guide endo-therapy accessories that are indicated to fit within a 2.0 mm inside diameter working channel.

In some embodiments, the device 1400 includes a luer lock mechanism 1611 that is configured to allow a syringe connection to the steerable sheath 1420.

FIG. 16B and FIG. 16C show exploded views, from different respective viewpoints, of an exemplary device 1648 including an exemplary handle connection mechanism 1650 and the steerable sheath 1420. In some embodiments, the handle connection mechanism 1650 is similar to the handle connection mechanism 1610 other than as described hereinafter. In some embodiments, the handle connection mechanism 1650 includes a steering lever 1652 that is similar to the steering lever 1602 other than as described hereinafter. In some embodiments, the handle connection mechanism 1650 includes a pull wire locking shaft 1654 that is coupled to the steering lever 1652 and to the pull wire 1606, and operates in a manner similar to the pull wire locking shaft 1604 described above. In some embodiments, the handle connection mechanism 1650 includes a locking knob 1656 that is similar to the locking knob 1605 described above. In some embodiments, the handle connection mechanism 1650 includes a luer lock mechanism 1658 that is configured to allow a syringe connection to the steerable sheath 1420. In some embodiments, the handle connection mechanism 1650 includes a position locker 1660 having locking teeth 1662. In some embodiments, the position locker 1660 is coupled to the steering lever 1652 and slidable laterally with respect to the steering lever 1652. In some embodiments, the handle connection mechanism 1650 includes a pull/push wire protector 1664 that covers the flexible pull wire 1606 and allows the pull wire 1606 to be pushed and pulled without bending, thereby protecting the pull wire 1606 from fatigue failures.

FIG. 17C and FIG. 17D show a perspective view and a side view, respectively, of the device 1648 with the steering lever 1652 positioned so as to place the device 1648 in a released configuration. FIG. 17E shows a side view of the device 1648 with the steering lever 1652 positioned so as to place the device 1648 in an active configuration. FIG. 17F shows a perspective view of the device 1648 with the steering lever 1652 positioned so as to place the device 1648 in an active configuration and with the position locker 1660 positioned in an unlocked position. FIG. 17G shows a partial cutaway view of the device 1648 with the steering lever 1652 and the position locker 1660 positioned as shown in FIG. 17F. In the partial cutaway view of FIG. 17G, a portion of the steering lever 1652 has been omitted to show that, in the unlocked position, the locking teeth 1662 are positioned to the side of and do not engage the locking knob 1656, thereby enabling the steering lever 1652 to be moved. FIG. 17H shows a perspective view of the device 1648 with the steering lever 1652 positioned so as to place the device 1648 in an active configuration and with the position locker 1660 positioned in a locked position. FIG. 17I shows a partial cutaway view of the device 1648 with the steering lever 1652 and the position locker 1660 positioned as shown in FIG. 17H. In the partial cutaway view of FIG. 17I, a portion of the steering lever 1652 has been omitted to show that, in the locked position, the locking teeth 1662 engage the locking knob 1656, thereby preventing the steering lever 1652 from moving.

In some embodiments, the steerable sheath 1420 includes radiopaque markers 1609. In some embodiments, the radiopaque markers 1609 are positioned in a pattern. In some embodiments, the pattern of the radiopaque markers 1609 includes differently sized rings extending around the steerable sheath 1420. In some embodiments, the pattern of the radiopaque markers 1609 includes rings irregularly spaced along the steerable sheath 1420. In some embodiments, the pattern of the radiopaque markers 1609 includes differently shaped rings extending around the steerable sheath 1420 in such a way that the 3D curvature of the steerable sheath 1420 can be identified from a single plane X-Ray snapshot.

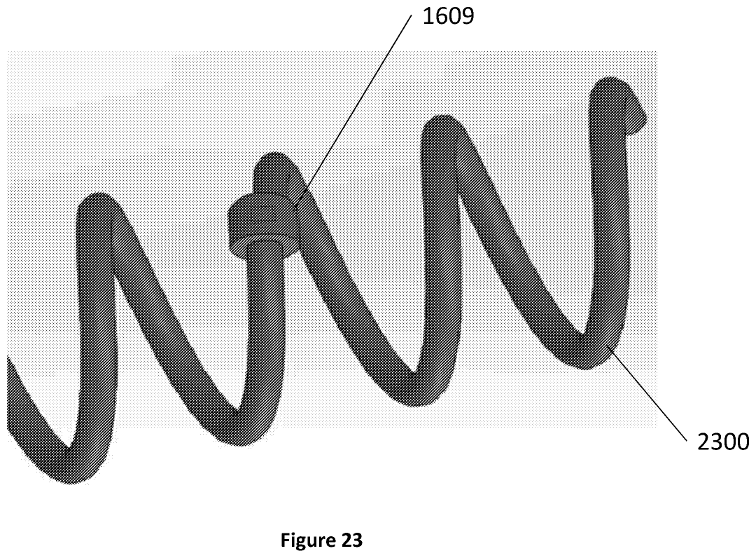

In some embodiments, the pattern of the radiopaque markers 1609 allows the derivation of the position of the sheath and its tip, including the roll, in six degrees of freedom from a single fluoroscopic image. In some embodiments, the pattern includes multiple markers 1609 attached along a braid that extends helically along the steerable sheath 1420. FIG. 23 shows an exemplary braid 2300 along which one of the radiopaque markers 1709 is positioned. It will be apparent to those of skill in the art that, while FIG. 23 shows the braid 2300 with one of the radiopaque markers 1709, this is only illustrative, and a practical implementation will include several of the radiopaque markers 1609. In some embodiments, the radiopaque markers are attached to the braid 2300 at predetermined distances, thereby forming a 3D structure of points that are not within the same plane. In some embodiments, knowing the 3D configuration of at least four markers 1609 allows estimation a pose of the sheath with six degrees of freedom. In some embodiments, use of less than four markers does not, on its own, provide a unique pose, but a unique pose can be determined by compensating with other sources of information. In some embodiments, knowledge of the pose (e.g., location and orientation) of the sheath in real time is helpful as this knowledge guidance to a physician as to how to manipulate a tool in order to locate it near a target. In some embodiments, such guidance may include directions to push and/or pull a sheath and change the tip orientation by rotating the sheath.

In some embodiments, a steerable sheath includes a central lumen, a handle to maneuver a catheter inside the body, at least one pull wire, a mechanism configured to displace the at least one pull wire, and a radiopaque pattern on the steerable sheath.

In some embodiments, movement of the at least one pull wire can be controlled by an electrical motor. In some embodiments, the electrical motor is integrated inside the handle. In some embodiments, the electrical motor is attached to the handle. In some embodiments, the motor is controllable by controls located on the handle. In some embodiments, the controls located on the handle include at least one of buttons and/or a joystick. In some embodiments, the motor is controllable by a controller located within the handle and configured to receive instructions from an internal device. In some embodiments, the controller is configured to receive instructions through a wired connection. In some embodiments, the controller is configured to receive instructions through a wireless connection. In some embodiments, the handle includes a power source. In some embodiments, the handle includes a connection to an external power source. In some embodiments, the handle includes a battery. In some embodiments, the handle includes a power storage element that is configured to be charged wirelessly.

In some embodiments, a method is provided for feedback loop navigation and guidance of the steerable traceable catheter along the planned pathway inside the body cavity. In some embodiments, in order to accurately navigate a steerable traceable instrument to a desired position inside a moving and dynamically changing body cavity (e.g., the structure of the bronchial airways), an exemplary embodiment provides real-time guidance in a continuous feedback loop to the user. In such an embodiment, real-time augmented imaging, such as augmented fluoroscopy, acts as a real-time navigation modality. In some embodiments, a live fluoroscopy image provides information regarding the position of the instrument being navigated relative to procedural augmented information. In some non-limiting examples, such procedural augmented information can include a highlighted target area, pathways, bifurcations, adjacent airways, and/or blood vessels, any of which can used to provide guidance to instrument positioning. In some embodiments, through the use of such guidance, simple instruments can be operated from outside the patient's body using a push and torque method and, optionally, steering the distal tip area of an instrument. In some embodiments, operation of an instrument is manual. In some embodiments, operation of an instrument is motorized (for example, using a robotic arm). In some embodiments, a continuous feedback loop improves the accuracy of target localization and visualization on an augmented image through feeding a true target location (e.g., an actual location as opposed to a calculated location) into a system providing augmented image data as additional weighted data for use in registration between preoperative and intraoperative imaging modulates. In some embodiments, the true target location is periodically acquired by a technique such as, but not limited to tomographic lesion reconstruction obtained from X-Ray devices such as a C-Arm, computed tomography (“CT”), or cone beam computed tomography (“CBCT”), or reconstructed from ultrasonic image data acquired by a radial endobronchial ultrasound (“rEBUS”) probe.

In some embodiments, a torque (e.g., rolling of the instrument) can be partially or completely avoided by allowing partially steerable instrument tip operation in a single plane or allowing steerable instrument tip operation in all directions. In some embodiments, a single plane steerable mechanism can be implemented by using one or two pull wires inside the sheath wall. In some embodiments, an all-directional steerable mechanism can be implemented using four pull wires inside the sheath wall.

In some embodiments, guidance over the real-time imaging modality can be in a form such as that of the augmented overlay described in International Patent Application Publication No. WO/2015/101948.

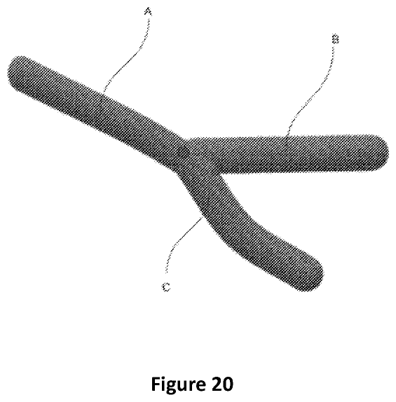

In some embodiments, navigating an instrument to a target requires navigating past a number of bifurcations on the way to the target. Each such bifurcation has its own corresponding three-dimensional anatomical structure. FIG. 20 shows the anatomical structure of a representative bifurcation. In some embodiments, when an instrument approaches the bifurcation through lumen A, there exists an optimal pose of an imaging device, such as a C-Arm, that will maximize the apparent angle between the projection of lumens B and C. In some embodiments, an exemplary method computes such an optimal pose and uses the optimal pose to navigate an instrument.

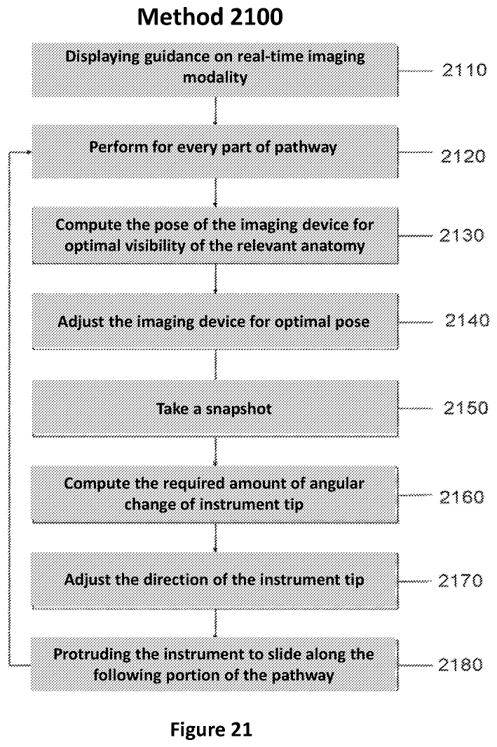

FIG. 21 shows a flowchart of such an exemplary method. In step 2110, navigation guidance is displayed on a real-time imaging modality (e.g., an intraoperative image). In step 2120, a navigation iteration is triggered for a given part (e.g., bifurcation) of a pathway to an area of interest. In step 2130, an optimal pose of an imaging device is calculated so as to provide an optimal view of the relevant anatomy (e.g., so as to maximize the apparent viewed angle between lumens B and C as shown in FIG. 20). In step 2140, the imaging device (e.g., an intraoperative imaging modality) is adjusted based on the optimal pose calculated in step 2130. In step 2150, at least one additional image is obtained following adjustment of the imaging device. In step 2160, a required amount of angular change of an instrument tip (e.g., of the distal end 1608 of the steerable sleeve 1420 as discussed above) is calculated. In step 2170, the direction of the instrument tip is adjusted (e.g., through the use of a pull wire 1606 to adjust the position of a distal end 1608 of a steerable sleeve 1420 as discussed above). In step 2180, the instrument is protruded to slide along the following portion of the pathway. Following step 2180, the method returns to step 2120 and subsequent iterations are triggered as needed until the instrument has reached the area of interest.

In some embodiments, a method of navigating an instrument (e.g., an endobronchial instrument) inside a body cavity includes the steps of: (1) displaying guidance on real-time imaging modality; and (2) performing, either dynamically or in a discrete way for each part of a pathway, the steps of: (a) if needed, recommending a change to the pose of an imaging device for optimal visibility of the relevant anatomy (such as a portion of the planned pathway or blood vessels in the proximity of the instrument), (b) recommending a change to the instrument tip flex angle to align with the following portion of pathway, and (c) protruding the instrument to slide along the following portion of the pathway.

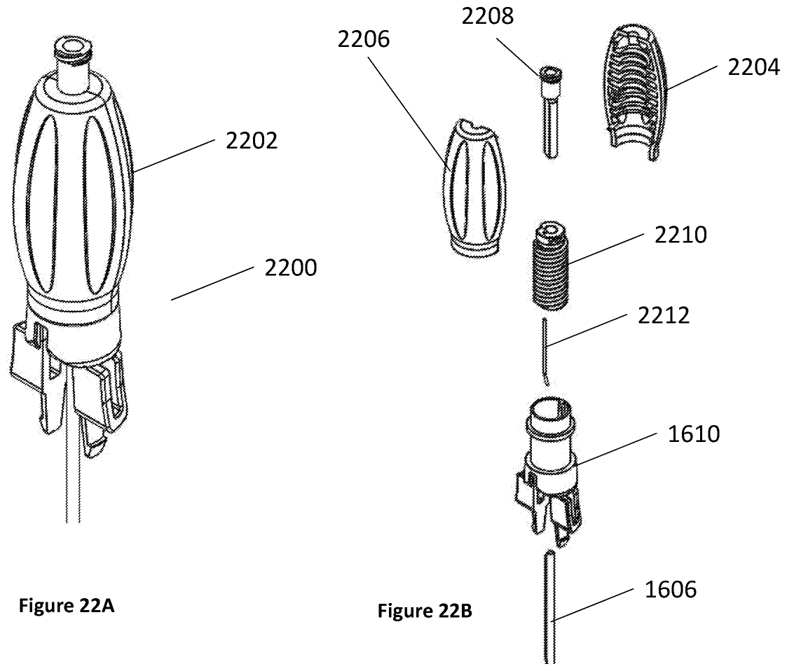

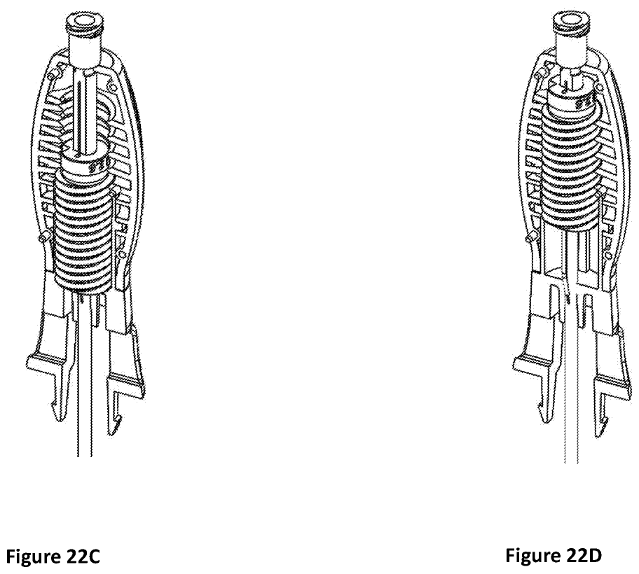

In some embodiments, the control portion of the steering mechanism is round. FIGS. 22A-22F illustrate elements and operation of an embodiment of a control portion that is round. FIG. 22A illustrates an exemplary control portion 2200. FIG. 22B illustrates an exploded view of the exemplary control portion 2200. In some embodiments, the control portion 2200 includes a handle 2202 formed from handle sides 2202, 2204. In some embodiments, the control portion 2200 includes a luer connection 2208 retained between the handle sides 2202, 2204. In some embodiments, the control portion 2200 includes a push/pull swivel 2210 retained between the handle sides 2202, 2204 and threadedly engaged with internal threads of the handle sides 2202, 2204. In some embodiments, the control portion 2200 includes a wire protector 2212. In some embodiments, as shown in FIGS. 22A and 22B, the pull wire 1606 is attached to the push/pull swivel 2208. In some embodiments, when the user rotates the handle 2202 of the control portion 2200, the push/pull swivel 2210 is caused to moves upward or downward along the handle connection mechanism 1610 due to the threaded engagement of the push/pull swivel 2210 with the handle 2202, thereby pulling on or releasing the pull wire 1606 and deflecting or releasing the distal end of the pull wire 1606 as described above. In some embodiments, the push/pull swivel 2210 is secured by a notch that is located inside a protrusion in the handle connection mechanism 1610, and thereby is only allowed to move longitudinally along the handle connection mechanism 1610 and is prevented from rotating with respect to the handle connection mechanism 1610. In some embodiments, the luer connection 2208 is coupled to the working channel of the sheath to enable an endotherapy tool to be introduced into the working channel of the sheath. In some embodiments, the wire protector 2212 covers the flexible pull wire and allows the pull wire to be pushed and pulled without bending, thereby preventing the pull wire from fatigue failures. FIG. 22C shows a section view of the handle 2200 with the push/pull swivel 2210 positioned at an end of the handle 2202 that is closest to the handle connection mechanism 1610, in which the pull wire 1606 is not pulled and the sheath is thereby positioned in a released position. FIG. 22D shows a section view of the handle 2200 with the push/pull swivel 2210 positioned at an end of the handle 2202 that is furthest from the handle connection mechanism 1610 (e.g., the handle 2202 has been rotated to cause the push/pull swivel 2210 to move along the handle 2202 from the position shown in FIG. 22C), in which the pull wire 1606 has been pulled toward the handle 2202 and the sheath is thereby positioned in an active position. In some cases, an instrument can be positioned in the area of interest but not in an optimal position. For example, a physician may be interested to have the tip of the instrument be directed exactly to the center of the target (e.g., the optimal position is directed exactly at the center of the target) in order to push a needle through the sheath and get inside the target. In some embodiments, the current 3D position of the instrument in relation to the target can be estimated using methods based on real-time imaging modality such as fluoroscopy. In some embodiments, the method of position estimation can include, but is not limited to, stereoscopic estimation from multiple planes as described in International Patent Application Publication No. WO/2017/153839. In addition, position estimation can include, but is not limited to, a computational tomography based method, such as CBCT or limited angle tomography, as described in International Patent Application Publication No. WO/2020/035730, the contents of which are incorporated herein by reference in their entirety. In some embodiments, the instrument includes radiopaque markers (e.g., positioned within the instrument, positioned on a surface of the instrument, etc.) for estimating a 3D instrument position.

In some embodiments, a method of dynamic iterative instrument alignment includes the steps of: (1) estimating a 3D position of the instrument in relation to the target using real-time imaging; (2) computing the amount of required roll and change in curvature of the tip from the estimated 3D position of the instrument in relation to the target; (3) changing the roll and the direction of the instrument tip; and (4) estimating a 3D position of the instrument in relation to the target using real-time imaging for verification and additional iteration (e.g., repetition of steps (2) and (3)) if needed.

In some embodiments, based on knowledge of the actual tip position and direction, a user is provided with one or more instructions of how to translate the deviation from the required trajectory to one or more instructions to a user. In some embodiments, such instructions can include, but are not limited to, (1) direction and amount of roll that needs to be applied to the instrument; (2) direction and amount of change of the steerable tip angle; (3) the amount of required movement along the longitudinal axis of the instrument; and/or (4) a qualitative indication that sufficient rotation has been achieved during gradual rotation of the instrument.

In some embodiments, to simplify the operation of tool controls such as the controls that change the deflection angle of the tip of a tool, such controls have discrete positions with predefined intervals or jumps (e.g., clicks) between the positions.

In some embodiments, for applications involving navigating without a wire, only a sheath is used. In some embodiments, a sheath with a central lumen kept open is useful for using additional endobronchial tools at the same time. For example, a radial endobronchial ultrasound (“rEBUS”) probe can be positioned within the sheath while navigating the sheath in order to quickly verify the locations inside the body without pulling it out for re-navigation.

As may be known to those of skill in the art, when using a steerable pre-curved catheter, such as a hollow sheath or an extended working channel, the placement of an instrument inside such a catheter may strengthen the tip of the catheter, thereby changing the direction of the tip achieved during navigation. The exemplary embodiments, practiced through the use of a steerable flexible catheter, improve on this deficiency through the ability to change the bending angle of the catheter as needed. For example, a needle can be kept within such a sheath while aligning the sheath toward the target. As a result, the needle can be easily extracted after the desired alignment is achieved.

While a number of embodiments of the present invention have been described, it is understood that these embodiments are illustrative only, and not restrictive, and that many modifications may become apparent to those of ordinary skill in the art. Further still, the various steps may be carried out in any desired order (and any desired steps may be added and/or any desired steps may be eliminated).

What is claimed is:

1. A device, comprising:
a sheath having a proximal end, a distal end opposite the proximal end, and a lumen extending through the sheath from the proximal end to the distal end, wherein the sheath is biased to a released position;
a pull wire extending along the sheath from the proximal end to the distal end and being coupled to the sheath at the distal end,
wherein the pull wire and the sheath are configured to cooperate such that pulling the pull wire toward the proximal end of the sheath causes the distal end of the sheath to assume an active position, and such that release of the pull wire causes the distal end of the sheath to return to the released position; and
a control portion coupled to the proximal end of the sheath and to the pull wire, wherein the control portion includes a control element operable to selectively pull the pull wire toward the proximal end of the sheath or to release the pull wire.

2. The device of claim 1, wherein the sheath comprises a plurality of radiopaque markers.

3. The device of claim 2, wherein the plurality of radiopaque markers are arranged in a pattern along the sheath.

4. The device of claim 1, wherein the sheath is sized and shaped to be received within a bronchoscope having a working channel with a diameter of 2.8 mm and to be able to receive within the sheath of the lumen an endo-therapy accessory that is configured to fit within a 2.0 mm inside diameter working channel.

5. The device of claim 1, wherein the released position is a straight position and the active position is a curved position.

6. The device of claim 7, wherein a curvature of the curved position is variable depending on an extent to which the pull wire is pulled toward the proximal end of the sheath.

7. The device of claim 1, wherein the control portion includes a lever operable by a user to pull the pull wire toward the proximal end of the sheath.

8. The device of claim 8, further comprising a locking mechanism operable by a user to lock the lever in a selected position.

9. The device of claim 1, wherein the control portion further comprises a luer lock configured to receive a syringe and to couple the syringe to the sheath.

10. The device of claim 1, further comprising a handle connection mechanism configured to couple the device to an applicator.

11. A method, comprising:
(1) providing a device including a sheath, a pull wire, and a control portion,
wherein the sheath includes a proximal end, a distal end opposite the proximal end, a lumen extending through the sheath from the proximal end to the distal end, wherein the sheath is biased to a released position, and wherein the sheath includes a plurality of radiopaque markers positioned along the sheath;
wherein the pull wire extends along the sheath from the proximal end to the distal end and is coupled to the sheath at the distal end,
wherein the pull wire and the sheath are configured to cooperate such that pulling the pull wire toward the proximal end of the sheath causes the distal end of the sheath to assume an active position, and such that release of the pull wire causes the distal end of the sheath to return to the released position, and
wherein the control portion is coupled to the proximal end of the sheath and to the pull wire, wherein the control portion includes a control element operable to selectively pull the pull wire toward the proximal end of the sheath or to release the pull wire;
(2) advancing the sheath into a body cavity of a patient so that the distal end of the sheath is positioned at a bifurcation within the body cavity;
(3) displaying a view of the sheath within the body cavity by a real-time medical imaging modality obtained with a medical imaging device;
(4) determining an optimal position of the distal end of the sheath to advance the sheath past the bifurcation;
(5) operating the control portion to position the distal end of the sheath at the optimal position; and
(6) advancing the sheath past the bifurcation.

12. The method of claim 11, further comprising the steps of:
determining an optimal pose of the medical imaging device to display the sheath and the bifurcation;
positioning the medical imaging device at the optimal pose; and
displaying an updated view of the sheath and the bifurcation,
wherein the optimal position is determined based on the updated view.

13. The method of claim 11, wherein the body cavity is a bronchial airway.

14. The method of claim 11, further comprising repeating steps (3), (4), (5), and (6) at a further bifurcation.

15. The method of claim 14, wherein steps (3), (4), (5), and (6) are repeated at further bifurcations until the distal end of the sheath reaches a target area.
